(19) United States
(12) Patent Application Publication
(10) Pub. No.: US 2025/0367393 A1
(43) Pub. Date: Dec. 4, 2025

(54) TRACHEOSTOMY TUBE MONITORING ACCESSORY AND USES THEREOF

(71) Applicants: Children's National Medical Center, Washington, DC (US); UNIVERSITY OF OREGON, EUGENE, OR (US)

(72) Inventors: Julie SHERMAN, Palo Alto, CA (US); Keat Ghee ONG, Eugene, OR (US); Kaylee MEYERS, Bay City, MI (US); Ethan L. COOPER, Garnet Valley, PA (US); Arsalan SIDDIQUI, Greenbelt, MD (US); Noah JAGDMAN, Abingdon, MD (US); Shahmeel NASEEM, Ellicott City, MD (US); Tuan Ishaque Aqeel MUTHALIFF, Burtonsville, MD (US)

(73) Assignees: Children's National Medical Center, Washington, DC (US); UNIVERSITY OF OREGON, EUGENE, OR (US)

(21) Appl. No.: 19/223,580

(22) Filed: May 30, 2025

Related U.S. Application Data
(60) Provisional application No. 63/654,838, filed on May 31, 2024.

(51) Int. Cl. A61M 16/00; A61M 16/04

(52) U.S. Cl. CPC A61M 16/024; A61M 16/0003; A61M 16/0051; A61M 16/0402; A61M 2205/18; A61M 2205/3306; A61M 2205/3327; A61M 2205/3592; A61M 2205/50; A61M 2205/7536; A61M 2230/40

(57) ABSTRACT

A device for detecting tracheostomy tube placement, including a housing having an inner wall forming a cylindrical channel through the housing, the cylindrical channel having a first opening and a second opening, a carbon dioxide sensor embedded in a cutout in the inner wall of the housing, a moisture filter removably affixed to the inner wall, and processing circuitry embedded in the housing and configured to determine a level of carbon dioxide in airflow through the cylindrical channel based on data from the carbon dioxide sensor, generate a waveform representing carbon dioxide levels in the airflow through the cylindrical channel, and determine a placement state of a tracheostomy tube that is removably coupled to the first opening of the cylindrical channel based on analysis of the waveform representing the carbon dioxide levels in the airflow, analysis of the waveform considering a physiological factor of the patient having the tracheostomy tube.

### CROSS-REFERENCE TO RELATED APPLICATIONS

The present application claims priority to U.S. Provisional Application No. 63/654,838, filed May 31, 2024, which is incorporated herein by reference in its entirety for all purposes.

### BACKGROUND

### Field of the Disclosure

The present disclosure pertains to securement devices for securing a tube, such as a medical catheter which is inserted into the body for passage of fluids into or out of the body.

### Description of the Related Art

Tracheostomy tubes are life-sustaining medical devices used to maintain an airway and facilitate breathing in individuals with respiratory problems. About 20% of tracheostomized pediatric patients experienced an emergency complication involving their tracheostomy tube due to accidental decannulation or obstruction events which may result in permanent neurological damage or death if not mitigated quickly enough.

The foregoing “Background” description is for the purpose of generally presenting the context of the disclosure. Work of the inventors, to the extent it is described in this background section, as well as aspects of the description which may not otherwise qualify as prior art at the time of filing, are neither expressly nor impliedly admitted as prior art against the present invention.

### SUMMARY

The foregoing paragraphs have been provided by way of general introduction and are not intended to limit the scope of the following claims. The described embodiments, together with further advantages, will be best understood by reference to the following detailed description taken in conjunction with the accompanying drawings.

In one embodiment, the present disclosure is related to a device for detecting tracheostomy tube placement, comprising a housing having an inner wall forming a cylindrical channel through the housing, the cylindrical channel having a first opening and a second opening; a carbon dioxide sensor embedded in a cutout in the inner wall of the housing; a moisture filter removably affixed to the inner wall; and processing circuitry embedded in the housing and configured to determine a level of carbon dioxide in airflow through the cylindrical channel based on data from the carbon dioxide sensor, generate a waveform of carbon dioxide levels in the airflow through the cylindrical channel, and determine a state of a tracheostomy tube that is removably coupled to the first opening of the cylindrical channel based on the waveform of carbon dioxide levels in the airflow.

In one embodiment, the present disclosure is related to device for detecting tracheostomy tube placement, comprising a housing having an inner wall forming a cylindrical channel through the housing, the cylindrical channel having a first opening and a second opening; a carbon dioxide sensor embedded in the inner wall of the housing; and processing circuitry embedded in the housing and configured to determine a level of carbon dioxide in airflow through the cylindrical channel based on data from the carbon dioxide sensor, generate a waveform of carbon dioxide levels in the airflow through the cylindrical channel, and determine a state of a tracheostomy tube that is removably coupled to the first opening of the cylindrical channel based on the waveform of carbon dioxide levels in the airflow.

In one embodiment, the present disclosure is related to tracheostomy tube, comprising: a cannula; a flange at a proximal end of the cannula; a carbon dioxide sensor embedded in an inner wall of the cannula at the proximal end of the cannula; and a housing containing processing circuitry that is removably coupled to the flange, wherein the processing circuitry is configured to determine a level of carbon dioxide in airflow through the cannula based on data from the carbon dioxide sensor, generate a waveform of carbon dioxide levels in the airflow through the cannula, and determine a state of the tracheostomy tube based on the waveform of carbon dioxide levels in the airflow.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1A is a carbon dioxide sensing device, according to one embodiment of the present disclosure;

FIG. 1B is a carbon dioxide sensing device, according to one embodiment of the present disclosure;

FIG. 2 is a carbon dioxide sensing device, according to one embodiment of the present disclosure;

FIG. 3 is a carbon dioxide sensing device, according to one embodiment of the present disclosure;

FIG. 4 is a carbon dioxide sensing device, according to one embodiment of the present disclosure;

FIG. 5A is a carbon dioxide sensing device, according to one embodiment of the present disclosure;

FIG. 5B is a carbon dioxide sensing device, according to one embodiment of the present disclosure;

FIG. 5C is a carbon dioxide sensing device, according to one embodiment of the present disclosure;

FIG. 5D is a carbon dioxide sensing device, according to one embodiment of the present disclosure;

FIG. 6 is a carbon dioxide sensing device, according to one embodiment of the present disclosure;

FIG. 7 is a carbon dioxide sensing device, according to one embodiment of the present disclosure;

FIG. 8 is a schematic for processing carbon dioxide sensor data, according to one embodiment of the present disclosure;

FIG. 9 is a user interface for carbon dioxide sensing, according to one embodiment of the present disclosure;

FIG. 10 is an illustration of carbon dioxide level waveforms, according to one embodiment of the present disclosure;

FIG. 11 is an illustration of carbon dioxide level waveforms, according to one embodiment of the present disclosure;

FIG. 12 is an illustration of carbon dioxide level waveforms, according to one embodiment of the present disclosure;

FIG. 13 is an illustration of a carbon dioxide level waveform, according to one embodiment of the present disclosure;

FIG. 14 is an illustration of carbon dioxide level waveforms, according to one embodiment of the present disclosure;

FIG. 15 is an illustration of carbon dioxide level waveforms, according to one embodiment of the present disclosure;

FIG. 16 is an illustration of a carbon dioxide sensing device, according to one embodiment of the present disclosure.

### DETAILED DESCRIPTION OF THE INVENTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

In one embodiment, the present disclosure is directed to systems and methods for monitoring tracheostomy (trach) tube insertion for pediatric patients. Accidental decannulation occurs when the trach tube becomes partially or fully removed from the tracheal stoma due to severe coughing, failure in the tube securement, or patients dislodging the tubes themselves. Trach tube obstruction can result from mucus occlusion or false passages when the trach tube is accidentally inserted into the pre-tracheal space. These issues are more common with pediatric patients due to smaller trach tube airway sizes and absence of inner canula placement. When a trach tube is decannulated or obstructed, cardiopulmonary distress and hypoxia begin immediately, and the airway must be re-established within 3-5 minutes to prevent permanent neurological damage or death. Infants and children with trach tubes can have difficulty vocalizing because air bypasses the vocal cords through the tubes, which makes it difficult for them to alert caregivers to issues with their trach tubes.

In one embodiment, the systems and methods can monitor trach tubes that are used without full-time artificial ventilation. Ventilators can include built-in sensors to detect breathing complications. However, the systems presented herein can be used to monitor breathing of patients who can breathe independently through trach tubes. Typical monitoring approaches rely on visual inspection and pulse oximetry. However, these techniques are inefficient and require continuous supervision. Caregivers may not immediately detect trach tube issues through visual inspection.

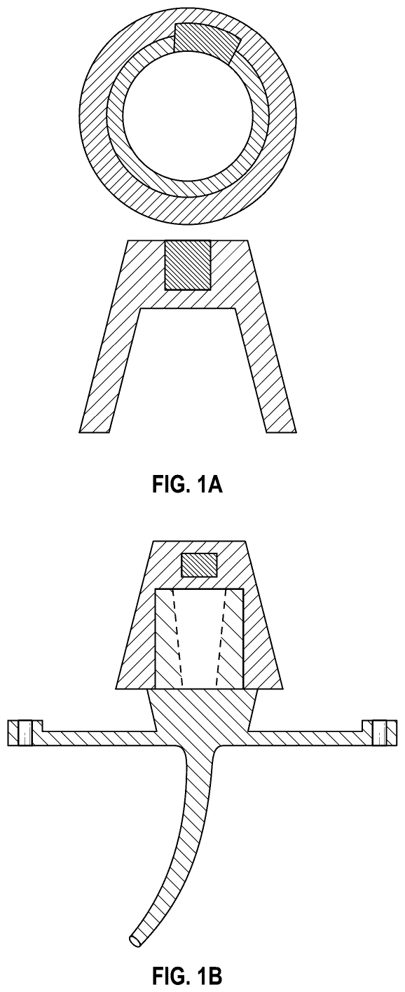

In one embodiment, the present disclosure is directed to a carbon dioxide sensor (sensing system, sensing device, assembly) that can be attached to a pediatric trach tube for real-time detection of trach tube issues. In one embodiment, the carbon dioxide sensing system can be embedded in a pediatric trach tube with a reusable electronics housing. FIG. 1A is an illustration of a carbon dioxide sensing device according to one embodiment. The body of the device can include a housing having inner walls that define a channel. The device can be inserted between the trach tube that is inserted into the patient and another trach tube attachment, such as a heat moisture exchanger or a voice valve. The channel can be aligned with the trach tube so that air can pass through the channel and to the attachment. In one embodiment, the channel can be approximately cylindrical. In one embodiment, the inner walls of the housing can be sloped so that a first opening of the channel is narrower than the opposite second opening of the channel. In one embodiment, the housing can include a carbon dioxide sensor and electronics as illustrated in the top view of FIG. 1A. In one embodiment, the carbon dioxide sensor can be embedded in the wall of the channel and facing the channel to monitor air that passes through the channel. The carbon dioxide sensor can acquire data from airflow that passes tangentially across (over) the sensor rather than in a mainstream configuration. In one embodiment, the carbon dioxide sensor can detect carbon dioxide that diffuses across the channel to the sensor. In one embodiment, the diffusion can be affected by respiration pressure, which is age-dependent. The carbon dioxide sensor can be a medical-grade sensor that detects airflow using a pyroelectric infrared gas sensor, e.g., a USE-QGSEAC82180 sensor. In one embodiment, the inner wall of the housing can include a cutout window to hold the carbon dioxide sensor and allow light passage/detection between the carbon dioxide sensor and the airflow. For example, the window can be approximately 4 millimeters (mm) by 6 millimeters (mm). In one embodiment, the carbon dioxide sensor can be a low-power sensor. The carbon dioxide sensor can detect exhaled carbon dioxide through the trach tube to monitor whether the patient is properly “breathing” through the trach tube. Issues with the trach tube will result in decreased and/or non-standard levels of carbon dioxide.

FIG. 1B is an illustration of the carbon dioxide sensing device attached to the trach tube according to one embodiment. In one embodiment, the inner walls of the housing can be lined with a soft material. The carbon dioxide sensor can be embedded in the soft material of the inner wall. In one embodiment, the carbon dioxide sensor can be located at an opening of the channel. In one embodiment, the housing can be a biocompatible material. For example, the housing can be 3D-printed using polyvinyl.

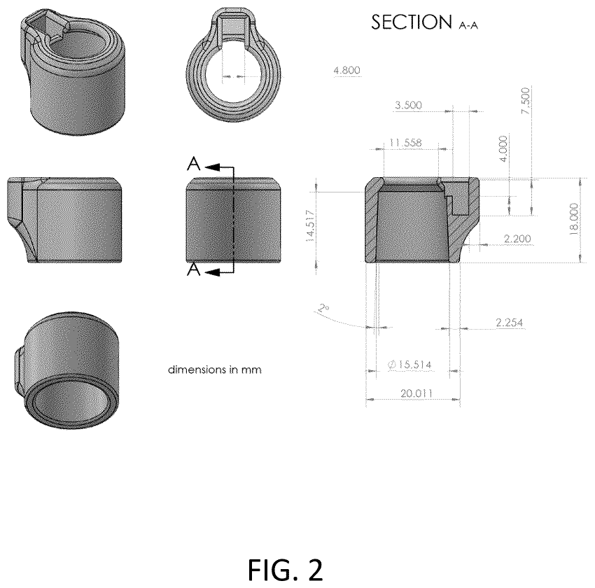

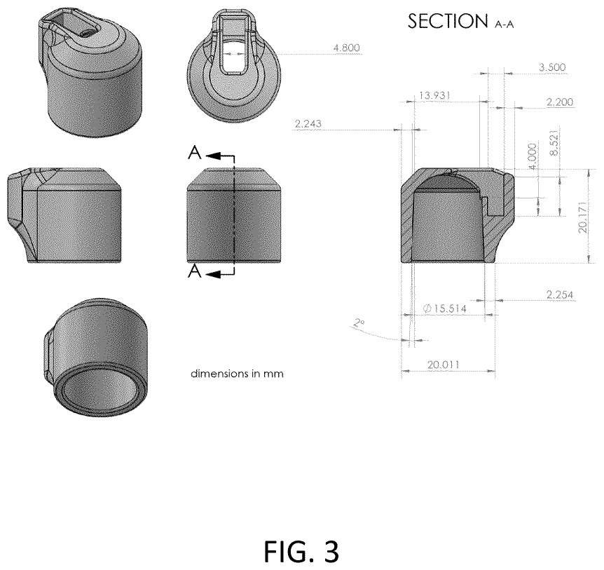

FIG. 2 is an illustration of a carbon dioxide sensing device according to one embodiment. In one embodiment, the housing can include a flange for holding the embedded carbon dioxide sensor and associated electronics. FIG. 3 is an illustration of a carbon dioxide sensing device according to one embodiment. The housing can include a flange to hold electronics. In one embodiment, one end (opening) of the housing can include a cover or cap to narrow the opening. In one embodiment, the cap can be shaped to fit concentrically into the channel. In one embodiment, the cap can be removable. In one embodiment, the cap can narrow one of the openings for a tighter fit with the trach tube components.

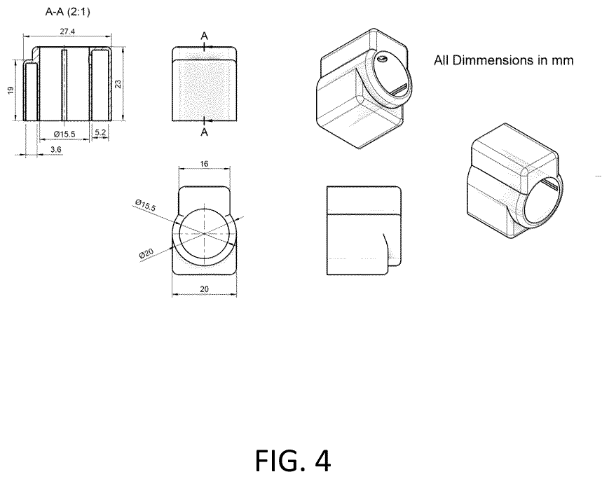

FIG. 4 is an illustration of a carbon dioxide sensing device according to one embodiment. In one embodiment, the housing can include a first flange and a second flange. The first flange and the second flange can be approximately on opposite sides of the housing. In one embodiment, the first flange can hold electronics. The first flange and the second flange can be the same size or different sizes. The flanges can provide handholds for easier handling of the device. In one embodiment, the housing can be approximately rectangular and can define an inner cylindrical channel.

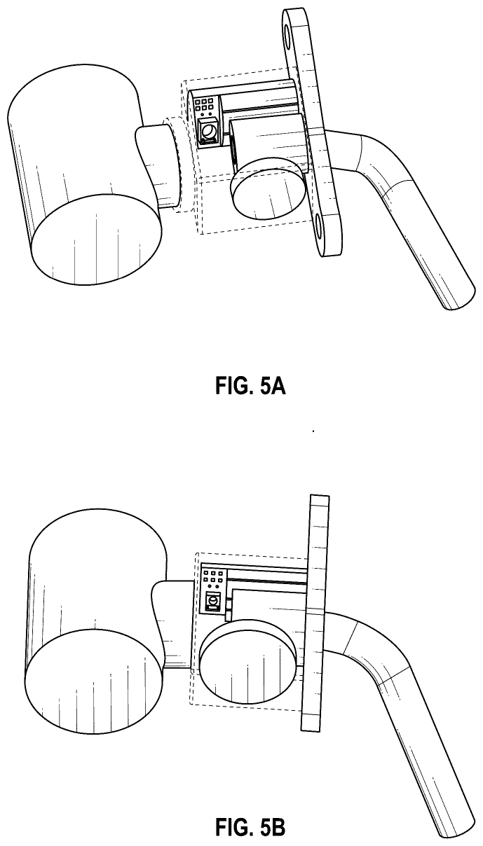

FIG. 5A is an illustration of the carbon dioxide sensing device assembled between a trach tube and a heat moisture exchanger according to one embodiment. In one embodiment, the carbon dioxide sensor in the housing of the carbon dioxide sensing device can be positioned at the distal opening of the channel, e.g. near the connection between the housing and the heat moisture exchanger. In one embodiment, the housing can further hold electronics to control the carbon dioxide sensor and collect and transmit data from the carbon dioxide sensor. For example, the housing can include a microcontroller having processing circuitry that is configured to receive and process data from the carbon dioxide sensor. In one embodiment, the microcontroller can be configured to use low-energy wireless communication. For example, the microcontroller can be configured to use a Bluetooth connection or WIFI connection to receive and/or transmit data such as data from the carbon dioxide sensor. In one embodiment, the housing can include a battery to power the carbon dioxide sensor and/or the microcontroller. In one embodiment, the electronics can be removably embedded or contained in the housing. In one embodiment, the electronics can be embedded in the housing with a cover or locking mechanism to prevent pediatric patients from removing components of the device.

FIG. 5B is an illustration of the carbon dioxide sensing device assembly with a larger battery. FIG. 5C is an illustration of the carbon dioxide sensing device assembly in an exploded view according to one embodiment. The two openings of the channel in the housing can be different sizes to fit the openings of the heat moisture exchanger and the tracheostomy tube, respectively. In one embodiment, each opening of the housing can fit with the corresponding component (trach tube or heat moisture exchanger) with a snap-fit (or press-fit) joint. FIG. 5D is an illustration of the carbon dioxide sensing device assembly when the trach tube is inserted into a pediatric patient (model).

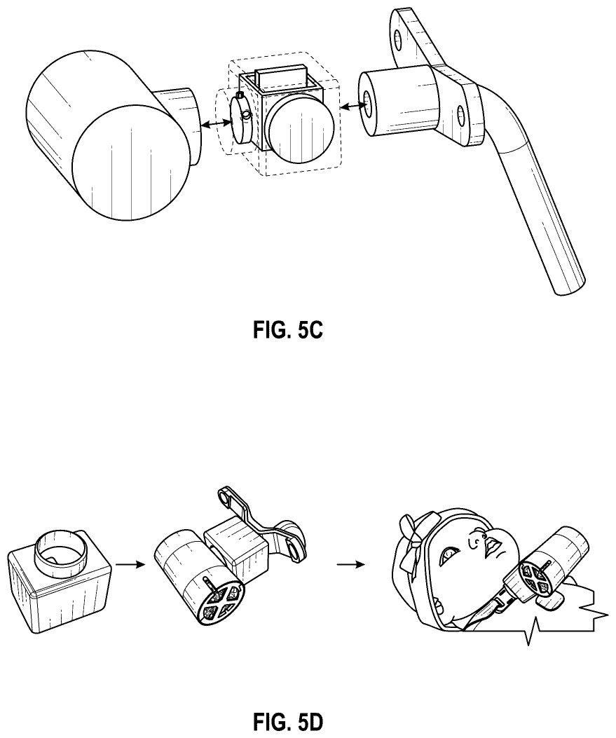

FIG. 6 is an illustration of the carbon dioxide sensing device assembly according to one embodiment. The carbon dioxide sensing device can include a liner (lining) along the inner wall of the housing. In one embodiment, the liner can be a disposable liner to maintain airway hygiene and facilitate device cleaning. In one embodiment, the liner can be a moisture barrier (e.g., moisture-absorbing) or filter to protect the embedded electronics (sensors, circuitry) in the housing from humidity and condensation. In one embodiment, the carbon dioxide sensing device can include a humidity sensor. The humidity sensor can detect moisture within the channel so that the carbon dioxide sensing device can alert a user when the disposable liner needs to be changed to avoid damage to the electronics.

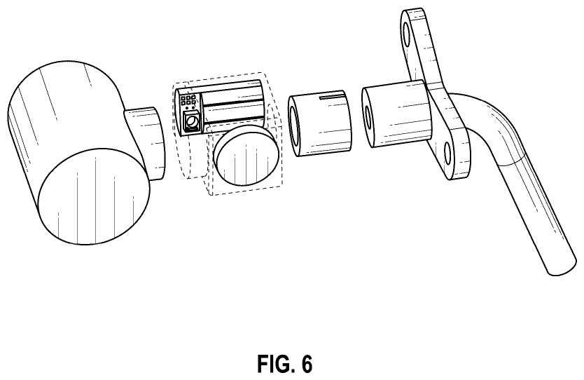

In one embodiment, the carbon dioxide sensing device can be embedded in a trach tube. FIG. 7 is an illustration of a trach tube including an embedded carbon dioxide sensor according to one embodiment. The carbon dioxide sensor can be embedded in an inner wall of a cannula of the trach tube itself to monitor airflow through the trach tube. The carbon dioxide sensor can be located outside of the body when the trach tube is inserted. In one embodiment, the carbon dioxide sensor can be located at a proximal end of the cannula, e.g., near the flange at the proximal end. In one embodiment, the electronics associated with the carbon dioxide sensor—e.g. a microcontroller and battery—can be attached to the exterior of the trach tube. For example, the electronics can be contained in a single electronics housing and the electronics housing can be attached to a flange of the trach tube faceplate as illustrated in FIG. 7. In one embodiment, the electronics housing can be attached to the flange using a strap or band that wraps around the flange. In one embodiment, the electronics housing can be attached to the ties that are used to secure the trach tube to the neck when inserted. In one embodiment, the electronics housing can be reusable and can be removed and attached to different trach tubes as needed. In one embodiment, the electronics can be connected to the carbon dioxide sensor via a wired connection. For example, the trach tube can include a small port through which to run the wired connection.

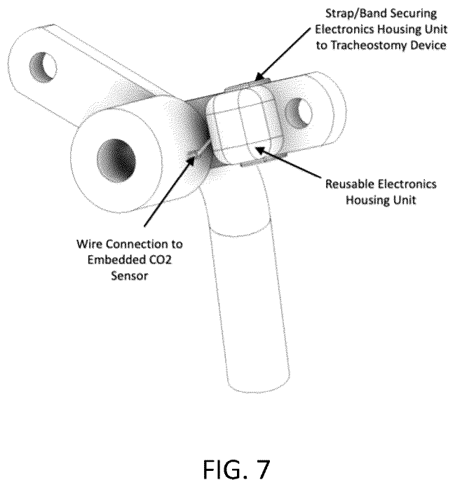

FIG. 8 is a schematic of data acquisition and transmission via the carbon dioxide sensing device according to one embodiment of the present disclosure. The microcontroller can acquire data from the carbon dioxide sensor and can process the data to determine the carbon dioxide levels in the trach tube. When the trach tube is inserted correctly, the carbon dioxide waveform is cyclical and reaches a certain level. When the trach tube is not inserted correctly, the carbon dioxide waveform has a different appearance. For example, when decannulation or full blockage occurs, the patient is no longer exhaling through the trach tube, and the carbon dioxide levels can fall to zero or a minimal amount. When partial mucus obstruction occurs, the waveform is distorted and resembles a sawtooth wave more than a square wave because the patient cannot exhale fully through the trach tube.

In one embodiment, signal analysis can be applied to the carbon dioxide sensor data in order to determine a state of the trach tube. The state of the trach tube can include, but is not limited to, correct insertion, incorrect insertion, decannulation, or blockage. The signal analysis can include, but is not limited to, thresholding techniques (e.g., amplitude thresholding, Fourier analysis) and signal shape characterization and analysis. As an example, thresholding can be used to detect decannulation and complete blockages, while signal shape analysis can be used to identify obstruction severity. In one embodiment, the signal analysis can include a machine learning approach. For example, a short-term memory neural network can be trained to receive carbon dioxide sensor data as an input and output a characterization of the trach tube insertion (e.g. properly inserted, decannulation, obstruction) as an output. In one embodiment, the signal analysis can be performed by the carbon dioxide sensor device. In one embodiment, the signal analysis can be performed by a user device as described herein.

In one embodiment, the microcontroller can transmit the carbon dioxide sensor data via a wireless connection to a receiver. In one embodiment, the receiver can be connected to or embedded in a user device such as a mobile phone, computer, tablet, etc. The user device can display the carbon dioxide sensor data. In one embodiment, the user device can generate a visual, audio, or tactile alert based on the carbon dioxide sensor data. For example, if the carbon dioxide waveform does not match the waveform of a correct tube insertion, the user device can determine a state of the trach tube based on the carbon dioxide waveform and can generate an alert indicating that the trach tube is not properly placed. In one embodiment, the alert can include a type of trach tube complication, such as decannulation or partial mucus obstruction, based on the carbon dioxide waveform.

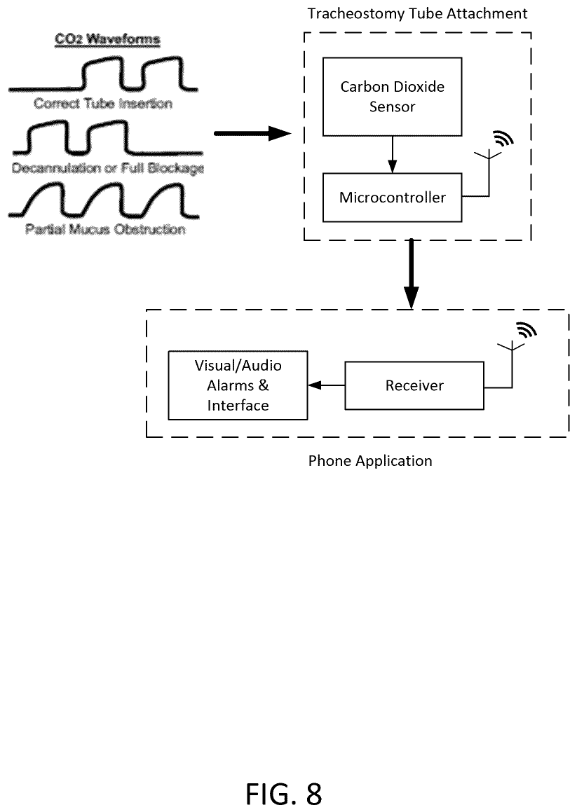

FIG. 9 is an illustration of a user interface displayed on a user device according to one embodiment of the present disclosure. In one embodiment, the user device can receive the carbon dioxide sensor data from the carbon dioxide sensor via an application and can display the carbon dioxide sensor data as a waveform of carbon dioxide levels over time. In one embodiment, the user device can receive instructions to generate an alert based on the carbon dioxide sensor data. In one embodiment, the user device can display information related to proper trach tube placement and/or mitigation of improper trach tube placement. In one embodiment, the user device can generate an alert when the user device is disconnected from the carbon dioxide sensor and/or based on a battery life of the carbon dioxide sensing device and/or the user device. In one embodiment, Wi-Fi protocols can be used to supplement a Bluetooth connection and provide an extended range of wireless connection between the carbon dioxide sensing device and the user device.

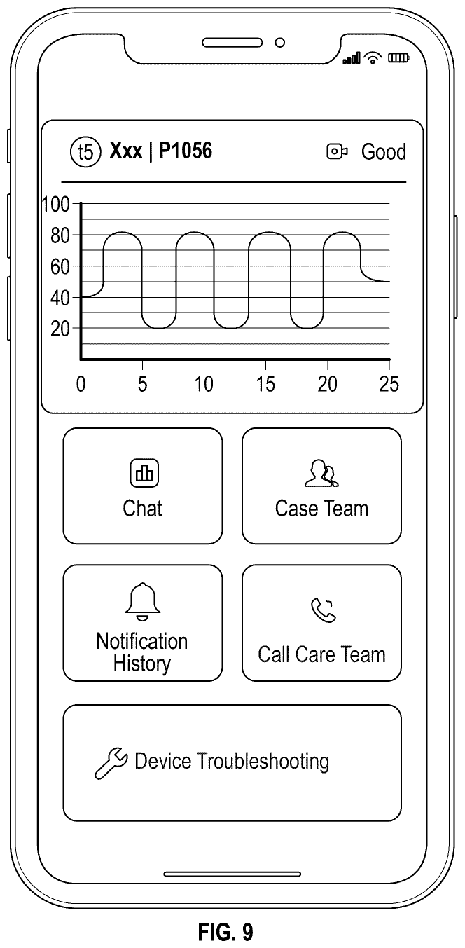

The carbon dioxide sensing device can be tested on a pediatric tracheal model to determine whether the device can accurately detect trach tube insertion and complications. The pediatric tracheal model can simulate pediatric respiration, including exhaled carbon dioxide, as well as trach tube complications and emergency events such as incorrect insertion, accidental decannulation, and mucus obstruction. FIG. 10 is an example of carbon dioxide levels based on data from the carbon dioxide sensing device according to one embodiment. The carbon dioxide sensing device can be used to monitor a simulated pediatric breathing model. In one embodiment, the sensor data can be smoothed, e.g. with a moving average filter. In one embodiment, the sensor data can be normalized to remove a baseline value. The frequency of the waveforms corresponds to age because respiration rate decreases with age. In one example, measured carbon dioxide levels using the carbon dioxide sensing device increase with age due to the effect of age-dependent respiratory pressure on the level of detected carbon dioxide.

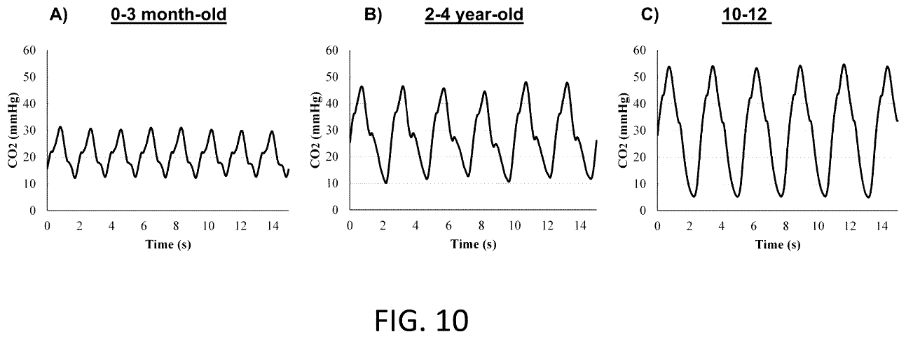

FIG. 11 is an example of carbon dioxide levels during a simulated correct trach tube placement and simulated incorrect trach tube placement (insertion into the pretracheal space) based on data from the carbon dioxide sensing device according to one embodiment. The simulated pediatric breathing model corresponds to a ventilator-independent toddler. Incorrect insertion results in a very low (ambient) level of carbon dioxide in the absence of exhaled breaths.

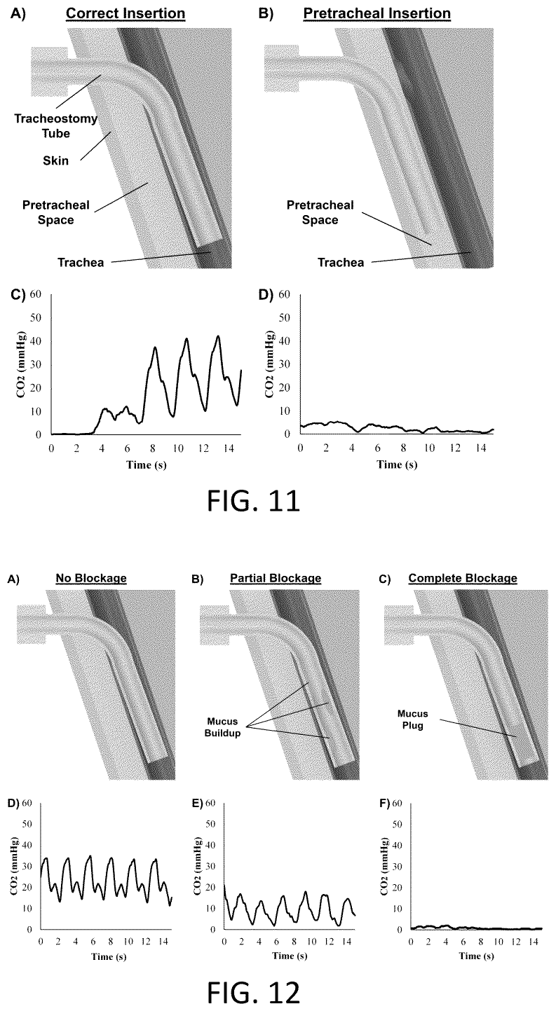

FIG. 12 is an example of carbon dioxide levels during a simulated no blockage, simulated partial mucus blockage, and simulated complete mucus blockage based on data from the carbon dioxide sensing device according to one embodiment. The simulated pediatric model corresponds to a ventilator-independent toddler. Mucus blockage results in lower levels of carbon dioxide and eventually an ambient level of carbon dioxide in complete blockage.

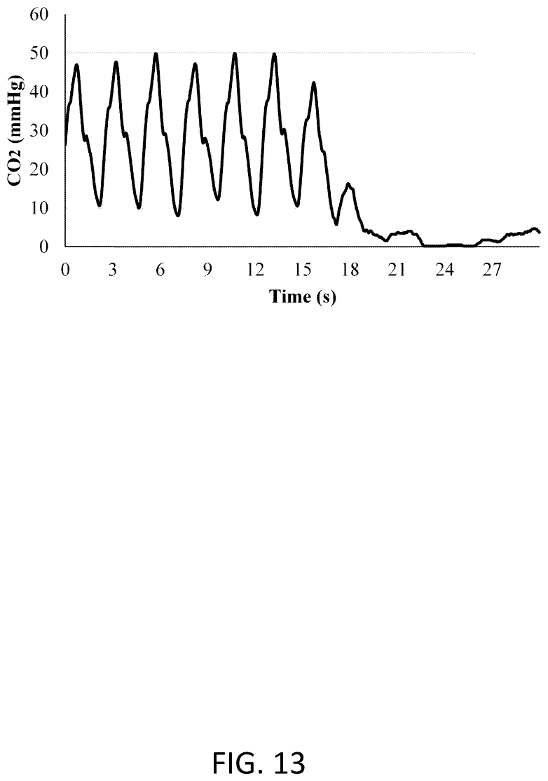

FIG. 13 is an example of carbon dioxide levels during a simulated decannulation based on data from the carbon dioxide sensing device according to one embodiment. The simulated pediatric breathing model corresponds to a ventilator-independent toddler. Initially, the trach tube is correctly placed for six breathing cycles. After the sixth breathing cycle, the simulated decannulation causes the carbon dioxide levels to decrease to an ambient level. In one embodiment, the carbon dioxide sensing device can trigger an alert based on the change in carbon dioxide levels/waveform when a complication occurs.

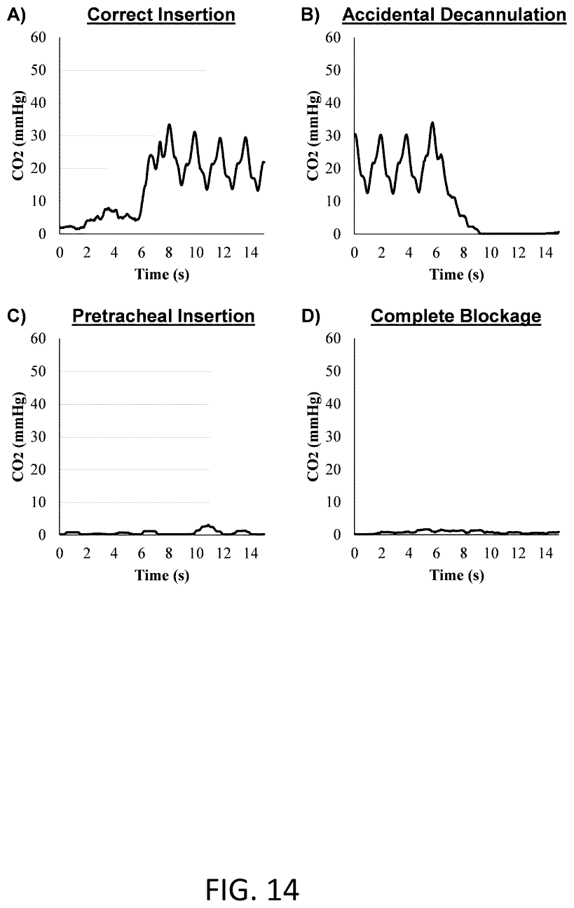

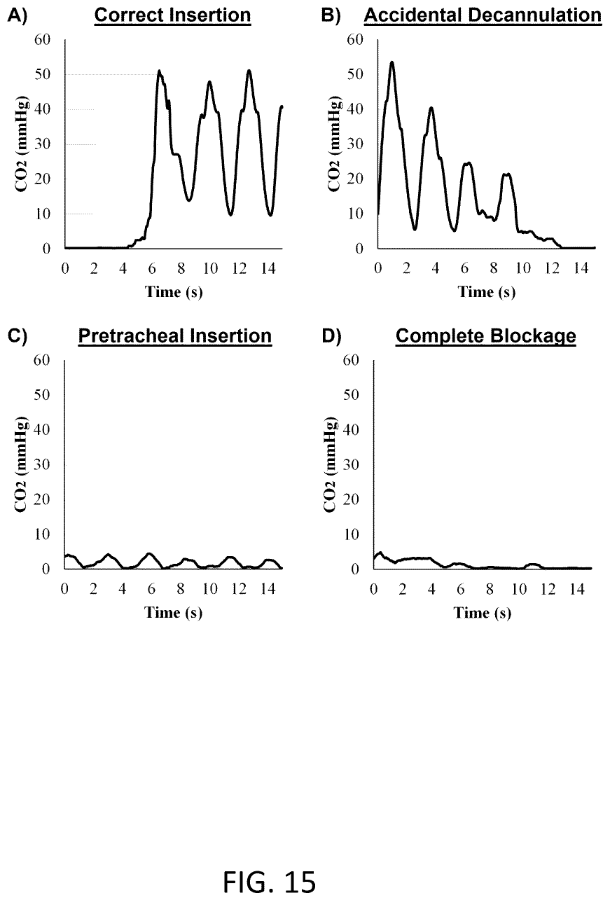

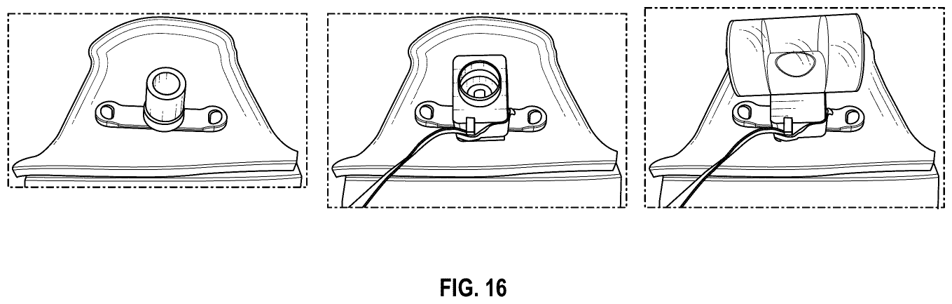

FIG. 14 is an example of carbon dioxide levels during simulated trach tube configurations for a neonatal patient based on data from the carbon dioxide sensing device according to one embodiment. FIG. 15 is an example of carbon dioxide levels during simulated trach tube configurations for a pediatric (10-12 years) patient based on data from the carbon dioxide sensing device according to one embodiment. The carbon dioxide sensing device is able to detect carbon dioxide waveforms at lower amplitudes (younger patients) and lower frequencies (older patients). FIG. 16 is an illustration of insertion of a trach tube with the carbon dioxide sensing device into a trachea model including simulated skin. In the first step, the trach tube can be inserted so that the flange and port are exposed. In the second step, a first opening of the carbon dioxide sensing device can be attached to the trach port, e.g. with a press-fit joint. Air leaving the trach tube can pass through the channel formed by the carbon dioxide sensing device and past the carbon dioxide sensor embedded in the inner wall of the device. In the third step, a heat moisture exchanger or other accessory can be attached to a second opening of the carbon dioxide sensing device. The trachea model can be connected to an aerosol generator, an auxiliary gas exchange cylinder acting as a balloon lung, and a breathing simulator to simulate the breathing of different types of pediatric patients with different trach tube configurations.

The carbon dioxide sensing device can be lightweight and compact so as to not interfere with trach tube functionality. In one embodiment, the device can be approximately 2.5 centimeters in length and weigh approximately 8.7 grams, which is a reduction in both length and weight from existing monitoring systems. The sensor and electronics can have low power consumption and can be reusable, resulting in increased longevity. In one embodiment, the electronics can be used for continuous monitoring for approximately 70 hours, which is an increase in battery life from existing monitoring systems. In one embodiment, the device can detect trach tube complications within approximately 20 seconds, which is faster than standard pulse oximeter techniques and gives caregivers more time to re-establish an airway before permanent injury or death occurs. The wireless communication between the carbon dioxide sensing device also enables the use of an application on a user device, which can provide quick and detailed alerts about different trach tube complications in both hospital and home settings.

While this specification contains many specific implementation details, these should not be construed as limitations on the scope of what may be claimed, but rather as descriptions of features that may be specific to particular embodiments.

Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system modules and components in the embodiments described above should not be understood as requiring such separation in all embodiments, and it should be understood that the described program components and systems can generally be integrated together in a single component or packaged into multiple components.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

Obviously, numerous modifications and variations are possible in light of the above teachings. It is therefore to be understood that within the scope of the appended claims, embodiments of the present disclosure may be practiced otherwise than as specifically described herein.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

1. A device for detecting tracheostomy tube placement, comprising:
a housing having an inner wall forming a cylindrical channel through the housing, the cylindrical channel having a first opening and a second opening;
a carbon dioxide sensor embedded in a cutout in the inner wall of the housing;
a moisture filter removably affixed to the inner wall; and
processing circuitry embedded in the housing and configured to
determine a level of carbon dioxide in airflow through the cylindrical channel based on data from the carbon dioxide sensor,
generate a waveform representing carbon dioxide levels in the airflow through the cylindrical channel, and
determine a placement state of a tracheostomy tube that is removably coupled to the first opening of the cylindrical channel based on analysis of the waveform representing the carbon dioxide levels in the airflow,
wherein the analysis of the waveform considers at least one physiological factor of a patient having the tracheostomy tube.

2. The device of claim 1, wherein the carbon dioxide sensor is an infrared sensor.

3. The device of claim 1, wherein the placement state of the tracheostomy tube includes a correct placement, an incorrect placement, a decannulation state, a partial obstruction, or a full obstruction.

4. The device of claim 1, further comprising a humidity sensor in contact with the inner wall of the housing.

5. The device of claim 1, wherein the first opening of the cylindrical channel is removably coupled to the tracheostomy tube with a press-fit joint.

6. The device of claim 1, wherein the processing circuitry is further configured to transmit the waveform representing carbon dioxide levels and the placement state of the tracheostomy tube to a user device via a wireless connection or transmit an alert to the user device based on the placement state of the tracheostomy tube via the wireless connection.

7. The device of claim 6, wherein the at least one physiological factor includes an age of the patient.

8. A device for detecting tracheostomy tube placement, comprising:
a housing having an inner wall forming a cylindrical channel through the housing, the cylindrical channel having a first opening and a second opening;
a carbon dioxide sensor embedded in the inner wall of the housing; and
processing circuitry embedded in the housing and configured to
determine a level of carbon dioxide in airflow through the cylindrical channel based on data from the carbon dioxide sensor,
generate a waveform representing carbon dioxide levels in the airflow through the cylindrical channel, and
determine a placement state of a tracheostomy tube that is removably coupled to the first opening of the cylindrical channel based on the waveform representing carbon dioxide levels in the airflow.

9. The device of claim 8, wherein the carbon dioxide sensor is an infrared sensor.

10. The device of claim 8, wherein the placement state of the tracheostomy tube includes a correct placement, an incorrect placement, a decannulation state, a partial obstruction, or a full obstruction.

11. The device of claim 8, further comprising a humidity sensor in contact with the inner wall of the housing.

12. The device of claim 8, wherein the first opening of the cylindrical channel is removably coupled to the tracheostomy tube with a press-fit joint.

13. The device of claim 8, wherein the processing circuitry is further configured to transmit the waveform representing carbon dioxide levels and the placement state of the tracheostomy tube to a user device via a wireless connection.

14. The device of claim 13, wherein the processing circuitry is further configured to transmit an alert to the user device based on the placement state of the tracheostomy tube via the wireless connection.

15. A tracheostomy tube, comprising:
a cannula;
a flange at a proximal end of the cannula;
a carbon dioxide sensor embedded in an inner wall of the cannula at the proximal end of the cannula; and
a housing containing processing circuitry that is removably coupled to the flange, wherein
the processing circuitry is configured to
determine a level of carbon dioxide in airflow through the cannula based on data from the carbon dioxide sensor,
generate a waveform representing carbon dioxide levels in the airflow through the cannula, and
determine a placement state of the tracheostomy tube based on the waveform of carbon dioxide levels in the airflow.

16. The tracheostomy tube of claim 15, wherein the carbon dioxide sensor is an infrared sensor.

17. The tracheostomy tube of claim 15, wherein the housing is removably coupled to the flange via a strap that is configured to wrap around the flange.

18. The tracheostomy tube of claim 15, wherein the placement state of the tracheostomy tube includes a correct placement, an incorrect placement, a decannulation state, a partial obstruction, or a full obstruction.

19. The tracheostomy tube of claim 15, wherein the processing circuitry is further configured to transmit the waveform representing carbon dioxide levels and the state of the tracheostomy tube to a user device via a wireless connection.

20. The tracheostomy tube of claim 19, wherein the processing circuitry is further configured to transmit an alert to the user device based on the placement state of the tracheostomy tube via the wireless connection.
